(19) United States
(12) Patent Application Publication
(10) Pub. No.: US 2023/0310812 A1
(43) Pub. Date: Oct. 5, 2023

(54) DOUBLE BALLOON VASCULAR OCCLUSION CATHETER

(71) Applicants: Benjamin R. Zambetti, Memphis, TN (US); Collin G. Howser, Nashville, TN (US)

(72) Inventors: Benjamin R. Zambetti, Memphis, TN (US); Collin G. Howser, Nashville, TN (US)

(21) Appl. No.: 17/708,975

(22) Filed: Mar. 30, 2022

(51) Int. Cl. A61M 25/10; A61B 17/12; A61M 25/01; A61M 25/00

(52) U.S. Cl. CPC A61M 25/1011; A61M 25/10184; A61B 17/12045; A61M 25/0108; A61M 25/0075; A61B 17/12136; A61M 2025/1052; A61M 2025/1013; A61M 2025/09125

(57) ABSTRACT

An apparatus and methods are provided for a double balloon vascular occlusion catheter. The double balloon vascular occlusion catheter includes a catheter for reaching an injury site within an artery. The catheter includes a central lumen for receiving a wire to guide the catheter to the injury site. A distal aspect of the double balloon vascular occlusion catheter includes proximal and distal balloons for being inflated on opposite sides of the injury site. A space between the balloons provides a blood occlusion segment of the catheter when the balloons are inflated. Inflation tubes and luer-locks at a proximal aspect of the double balloon vascular occlusion catheter enable inflation of the balloons. The double balloon vascular occlusion catheter may include a bidirectional lumen that causes unobstructed blood flow to bypass the blood occlusion segment.

### FIELD

Embodiments of the present disclosure generally relates to endovascular catheters. More specifically, embodiments of the disclosure relate to an apparatus and methods for a double balloon occlusion catheter for allowing proximal and distal control of a vascular injury through endovascular occlusion.

### BACKGROUND

Vascular injury is a common problem both in traumatic and iatrogenic settings. Traumatic vascular injuries account for significant morbidity and mortality with increased risk of limb loss and long-term repercussions associated with delayed repair. Similarly, iatrogenic injuries caused by vascular closure device failure or pseudoaneurysm formation after endovascular procedures can lead to significant blood loss and hemodynamic instability. While currently direct pressure is the standard of care followed by direct repair, this is cumbersome requiring medical personnel to apply pressure until an operating room can be prepared. Otherwise, a tourniquet is used with resultant ischemia of the distal extremity. In addition to this, not all vascular injuries are in compressible locations that can be managed with either manual compression or tourniquet application.

Resuscitative endovascular balloon occlusion of the aorta (REBOA) has been established as an accepted method of endovascular control for unknown central trauma, particularly useful with pelvic fractures and arterial bleeding. Recent studies of peripheral trauma have shown decreased morbidity and mortality with the use of endovascular control and stenting. Unfortunately, stenting has several risks that make it undesirable, whether that be young patients with high risk of in-stent stenosis or vessels not amenable to stenting. And while REBOA has been used for temporary control of central trauma, a device that allows proximal and distal endovascular control of the central and peripheral vasculature does not yet exist.

Therefore, a need exists for an apparatus providing endovascular control of vascular injuries until either definitive repair can be accomplished or concomitant lethal injuries have been managed. By allowing for early front-end control of arterial/venous bleeding, smaller incisions will be able to be used, other injuries will be able to be addressed, and blood loss will more quickly be controlled. It is contemplated that these factors will provide improved survival, improved limb salvage, decreased incision size, decreased wound infection, and less need for fasciotomies and complex wounds.

### SUMMARY

An apparatus and methods are provided for a double balloon vascular occlusion catheter. The double balloon vascular occlusion catheter includes a catheter for reaching an injury site within an artery. The catheter includes a central lumen for receiving a wire to guide the catheter to the injury site. A distal aspect of the double balloon vascular occlusion catheter includes proximal and distal balloons for being inflated on opposite sides of the injury site. A space between the balloons provides a blood occlusion segment of the catheter when the balloons are inflated. Inflation tubes and luer-locks at a proximal aspect of the double balloon vascular occlusion catheter enable inflation of the balloons. The double balloon vascular occlusion catheter may include a bidirectional lumen that provides unobstructed blood flow to bypass the blood occlusion segment.

In an exemplary embodiment, a vascular occlusion catheter comprises: a catheter for reaching an injury site within an artery; a proximal balloon for being inflated at a first side of the injury site; a distal balloon coupled for being inflated at a second side of the injury site; and a blood occlusion segment disposed between the proximal balloon and the distal balloon.

In another exemplary embodiment, the catheter includes a shunt that extends through the proximal balloon and the distal balloon to maintain blood flow through the artery. In another exemplary embodiment, the proximal balloon and the distal balloon are disposed near a distal aspect of the catheter with a space between the proximal balloon and the distal balloon to provide the blood occlusion segment of the catheter when the proximal balloon and the distal balloon are inflated. In another exemplary embodiment, one or more of the proximal balloon and the distal balloon include a marker for radiographic visualization.

In another exemplary embodiment, the catheter includes a central lumen for receiving a wire to guide the catheter to the injury site. In another exemplary embodiment, a luer-lock is disposed at a proximal aspect of the catheter for providing access to the central lumen for flushing and wire entry. In another exemplary embodiment, a first luer-lock and a second luer-lock are disposed near a proximal aspect of the catheter for respectively inflating the proximal balloon and the distal balloon.

In another exemplary embodiment, a first inflation tube is disposed in the central lumen and provides fluid communication between the first luer-lock and the proximal balloon. In another exemplary embodiment, a second inflation tube is disposed in the central lumen and provides fluid communication between the second luer-lock and the distal balloon. In another exemplary embodiment, a side port is disposed near a proximal aspect of the catheter and configured to facilitate flushing the central lumen and injecting contrast dye.

In another exemplary embodiment, the catheter includes proximal shunt perforations that are proximal of the proximal balloon and configured to allow blood to enter a central lumen of the catheter and exit through an open distal end of the catheter. In another exemplary embodiment, the catheter includes distal shunt perforations that are distal of the distal balloon and configured to allow blood to enter or exit a bidirectional lumen of the catheter. In another exemplary embodiment, the bidirectional lumen is concentric with a central lumen and provides fluid communication between the distal shunt perforations and the proximal shunt perforations. In another exemplary embodiment, the bidirectional lumen is configured to cause blood flow to bypass the blood occlusion segment.

In an exemplary embodiment, a method for vascular occlusion comprises: navigating a wire to an injury site within an artery; crossing the injury site with the wire; advancing a catheter over the wire to the injury site; positioning a proximal balloon at a first side of the injury site; positioning a distal balloon at a second side of the injury site; and inflating the proximal balloon and inflating the distal balloon to establish a blood occlusion segment at the injury site.

In another exemplary embodiment, positioning the proximal balloon and positioning the distal balloon include using radio-opaque marker to indicate the centers of the proximal balloon and the distal balloon within the artery. In another exemplary embodiment, inflating the proximal balloon and inflating the distal balloon includes causing unobstructed blood flow to bypass the blood occlusion segment. In another exemplary embodiment, causing unobstructed blood flow includes directing the blood flow through proximal shunt perforations into a central lumen of the catheter. In another exemplary embodiment, causing unobstructed blood flow includes directing the blood flow through a bidirectional lumen comprising the catheter. In another exemplary embodiment, directing the blood flow includes allowing blood to flow through proximal shunt perforations and distal shunt perforations that are in fluid communication with the bidirectional lumen.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an exemplary-use environment wherein a double balloon occlusion catheter is being used to shunt blood around a partial thickness injury of a subclavian artery, according to the present disclosure;

FIG. 2 illustrates an exemplary-use environment wherein a double balloon occlusion catheter is being used control bleeding into the retroperitoneum due to an injury to the iliac artery from a high stick for an angiogram;

FIG. 3 illustrates an exemplary embodiment of a double balloon occlusion catheter comprising two balloons at a distal aspect of the catheter with space between to provide a blood occlusion segment of the catheter, according to the present disclosure;

FIG. 4 illustrates an exemplary embodiment of a double balloon occlusion catheter that includes a port for injection of contrast for angiography and for flushing the catheter system in accordance with the present disclosure;

FIG. 5 illustrates an exemplary embodiment of a double balloon occlusion catheter that includes a proximal porous segment that shunts blood around an occluded segment to an open distal end of the catheter, according to the present disclosure;

FIG. 6 illustrates an exemplary embodiment of a double balloon occlusion catheter that includes a proximal porous segment and a distal porous segment that provide a bidirectional shunt of blood around an occluded segment of the catheter, in accordance with the present disclosure;

FIG. 7 illustrates a cross-sectional view of the catheter of FIG. 3, taken along line 7-7 according to the present disclosure; and

FIG. 8 illustrates a cross-sectional view of the catheter of FIG. 6, taken along line 8-8 according to the present disclosure.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the double balloon occlusion catheter and methods disclosed herein may be practiced without these specific details. In other instances, specific numeric references such as “first shunt,” may be made. However, the specific numeric reference should not be interpreted as a literal sequential order but rather interpreted that the “first shunt” is different than a “second shunt.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Vascular injury is a common problem both in traumatic and iatrogenic settings. A need exists for an apparatus providing endovascular control of vascular injuries until either definitive repair can be accomplished or concomitant lethal injuries have been managed. By allowing for early front-end control of arterial/venous bleeding, smaller incisions will be able to be used, other injuries will be able to be addressed, and blood loss will more quickly be controlled. It is contemplated that these factors will provide improved survival, improved limb salvage, decreased incision size, decreased wound infection, and less need for fasciotomies and complex wounds. Embodiments disclosed herein provide a double balloon occlusion catheter (hereinafter, “DBOC”) that enables proximal and distal control of a vascular injury through endovascular occlusion, and thus decreases potential blood loss.

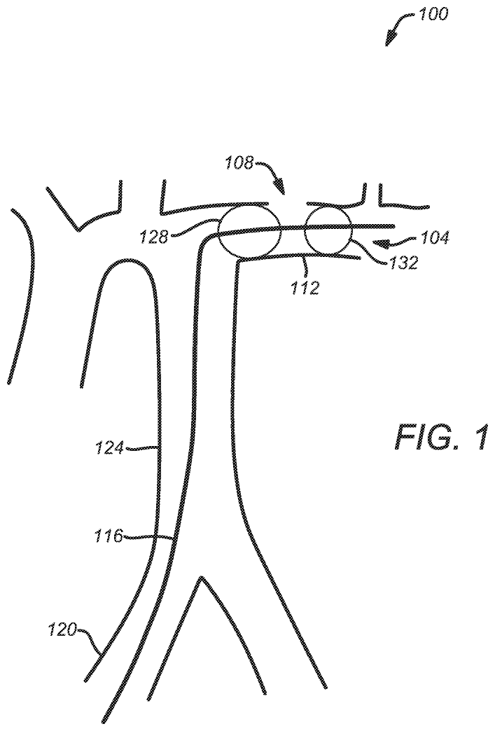

FIG. 1 illustrates an exemplary-use environment 100 wherein a DBOC 104 is being used to shunt blood around a partial thickness injury 108 of a subclavian artery 112, according to the present disclosure. As shown in FIG. 1, a catheter 116 is extended through the common iliac artery 120 and the aorta 124 such that the DBOC 104 is positioned at the site of the injury 108. The DBOC 104 includes a proximal balloon 128 and a distal balloon 132 that may be positioned on opposite sides of the injury 108, and then the balloons 128, 132 may be inflated to occlude blood flow to the injury 108. As described herein, a shunt is disposed within the catheter 116 and extends through the balloons 128, 132 to maintain blood flow through the subclavian artery 112 while the balloons 128, 132 are inflated to control hemorrhaging through the injury 108.

As will be appreciated, the DBOC 104 is configured for occlusion of major vascular structures for hemorrhage control. FIG. 1 highlights one application of the DBOC 104 by illustrating proximal and distal control of the subclavian artery, which is notoriously difficult to manage owing to its anatomical location and the multiple collateral blood supply through the circle of Willis.

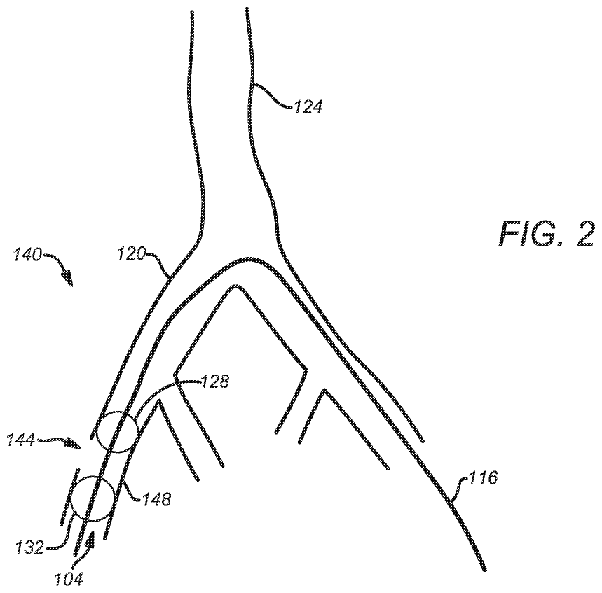

It should be recognized that the DBOC 104 can be deployed in any vessel and used to provide proximal and distal control of bleeding with an option of distal shunting. For example, FIG. 2 illustrates an exemplary-use environment 140 wherein the DBOC 104 is being used to control bleeding into the retroperitoneum due to an injury 144 to the external iliac artery 148 from a high stick for an angiogram. As shown in FIG. 2, the catheter 116 is extended through a contralateral groin stick, into the common iliac artery 120 and the external iliac artery 148 such that the DBOC 104 is positioned at the site of the injury 144. The proximal balloon 128 and the distal balloon 132 are positioned on opposite sides of the injury 144 and then inflated to occlude blood flow to the injury 144. As described herein, a shunt is disposed within the catheter 116 and extends through the balloons 128, 132 to maintain blood flow through the external iliac artery 148 while the balloons 128, 132 are inflated to control hemorrhaging through the injury 144.

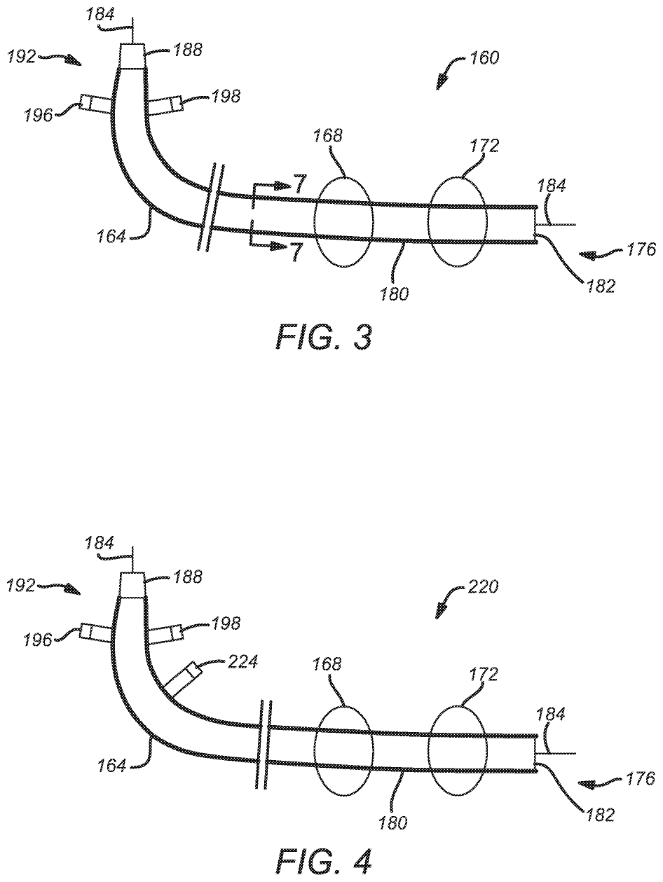

Turning, now, to FIG. 3, an exemplary embodiment of a DBOC 160 is illustrated according to the present disclosure. The DBOC 160 comprises a catheter 164, a proximal balloon 168, and a distal balloon 172. The proximal and distal balloons 168, 172 are disposed near a distal aspect 176 of the catheter 164 with a space between the balloons 168, 172 to provide a blood occlusion segment 180 of the catheter when the balloons 168, 172 are inflated. The catheter 164 includes a central lumen 182 that receives a wire 184. The wire 184 is removable and extends through the length of the catheter 164. The central lumen 182 may have a diameter that accommodates a 0.018″ wire 184 for smaller diameter catheters 164 or a 0.035″ wire 184 for larger size diameter catheters 164. A luer-lock 188 disposed at a proximal aspect 192 of the DBOC 104 provides access to the central lumen 182 for flushing and wire 184 entry.

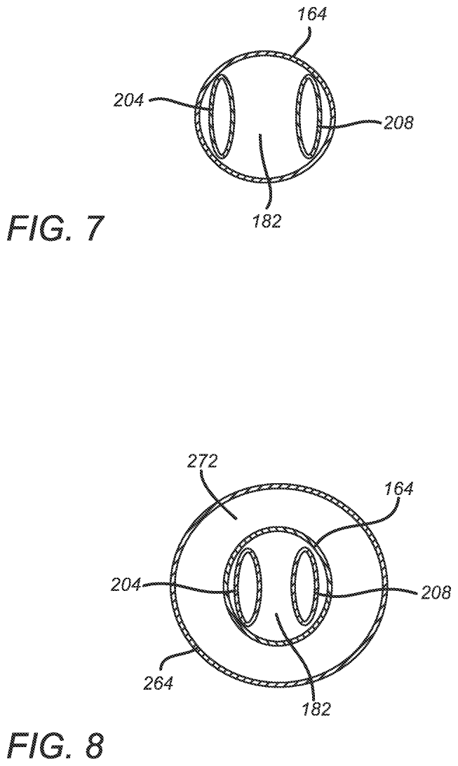

Moreover, the proximal aspect 192 includes a luer-lock 196 for inflating the proximal balloon 168 and a luer-lock 198 for inflating the distal balloon 172. As best shown in FIG. 7, an inflation tube 204 is disposed in the central lumen 182 and provides fluid communication between the luer-lock 196 and the proximal balloon 168. Similarly, fluid communication between the luer-lock 198 and the distal balloon 172 is provided by an inflation tube 208 that is disposed in the central lumen 182. It is contemplated that the balloons 168, 172 may be inflated by way of standard luer-lock syringes coupled with the luer-locks 196, 198. Further, in some embodiments, the central lumen 182 has a diameter that is large enough to allow the balloons 168, 172, while deflated, to be advanced over a wire to an injury site.

During operation, the DBOC 104 may be placed either percutaneously or through direct cutdown, in standard endovascular fashion. The catheter 164 may be advanced over the wire 184 once the wire 184 has crossed an injured segment of an artery. It is contemplated that each of the balloons 168, 172 includes a radio-opaque marker indicating the centers of the balloons 168, 172 within the artery. The DBOC 104 is superior to conventionally available options for endovascular control, particularly for young patients, as it does not require permanent stent placement. The DBOC 104 also provides proximal and distal control of a vessel as compared to currently available single balloon catheters. By providing the blood occlusion segment 180 between the balloons 168, 172, the DBOC 104 can be used instead of clamps for open procedures thus obviating a need for large incisions.

FIG. 4 illustrates an exemplary embodiment of a DBOC 220 according to the present disclosure. The DBOC 220 shown in FIG. 4 is substantially similar to the DBOC 160, shown in FIG. 3, with the exception that the DBOC 220 includes a side port 224 configured to facilitate flushing the catheter 164 and injecting contrast dye. In some embodiments, the side port 224 comprises a separate intraluminal catheter that enables an introduction of contrast and/or saline into the catheter 164.

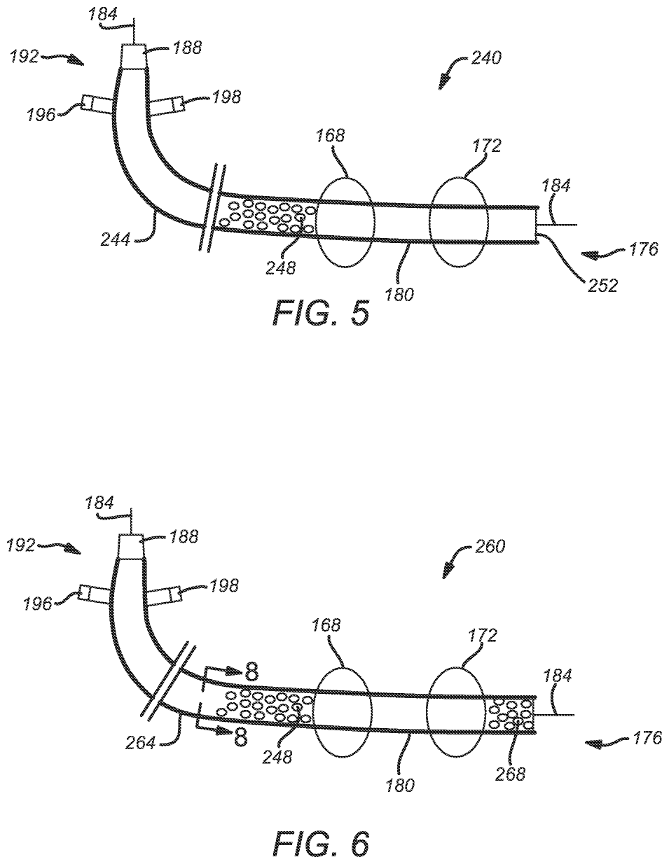

FIG. 5 illustrates an exemplary embodiment of a DBOC 240, according to the present disclosure. The DBOC 240 shown in FIG. 5 is substantially similar to the DBOC 160, shown in FIG. 3, with the exception that the DBOC 240 comprises a catheter 244 that includes proximal shunt perforations 248 that are proximal of the proximal balloon 168. The proximal shunt perforations 248 comprise a multiplicity of openings through the sidewall of the catheter 244 that allow blood to enter a central lumen 252 of the catheter 244 and exit through an open distal end of the catheter 244. As such, the proximal shunt perforations 248 and the central lumen 252 allow a continuation of blood flow that bypasses an occluded segment 180 between the balloons 168, 172. As will be appreciated, shunting blood around the occluded segment 180 advantageously decreases ischemia time to a distal extremity. It is contemplated that the DBOC 240, shown in FIG. 5, is particularly useful in settings of damage control operations or when other concurrent procedures are required, such as orthopedic fixation or laparotomy, or in a setting of prolonged interfacility transfer to a facility where definitive repair of the vascular injury can be performed.

FIG. 6 illustrates an exemplary embodiment of a DBOC 260, in accordance with the present disclosure. The DBOC 260 shown in FIG. 6 is substantially similar to the DBOC 240, shown in FIG. 5, with the exception that the DBOC 260 comprises a catheter 264 that includes distal shunt perforations 268 that are distal of the distal balloon 172. The distal shunt perforations 268 comprise a multiplicity of openings through the sidewall of the catheter 264 that allow blood to enter or exit a bidirectional lumen 272 of the catheter 264. As shown in FIG. 8, the bidirectional lumen 272 is concentric with a central lumen 182. As discussed with respect to FIG. 7, the central lumen 182 receives the wire 184 and carries an inflation tube 204 for the proximal balloon 168 and an inflation tube 208 for the distal balloon 172. The bidirectional lumen 272 provides fluid communication between the distal shunt perforations 268 and the proximal shunt perforations 248.

In particular, the proximal shunt perforations 248, the bidirectional lumen 272, and the distal shunt perforations 268 allow blood flow to bypass the occluded segment 180 between the balloons 168, 172 in either direction. It is contemplated that the DBOC 260, shown in FIG. 6, advantageously enables the catheter 264 to be routed from the ipsilateral groin to an extremity injury or in a setting of aortic work allowing distal perfusion, such as if used for resuscitation for a ruptured saccular thoracic aneurysm.

It is envisioned that any of the DBOCs 160, 220, 240, 260 disclosed hereinabove may comprise a single construct or may comprise multiple components that are assembled. In some embodiments, wherein the DBOC 160, 220, 240, 260 comprises a single construct, the DBOC 160, 220, 240, 260 may be made by way of molding, various casting techniques, or other suitable means. The catheters 164, 244, 264 may be sized on the French scale and may vary in size between 3 French to about 10 French. The size of the catheter 164, 244, 264 used will vary depending on the blood vessel to be treated. Further, the catheter 164, 244, 264 may have a length ranging between about 30 centimeters (cm) and about 120 cm. The catheter 164, 244, 264 may be made of a pliable polymer, such as, but not limited to, Pellethane, Teflon, silicon, and the like.

The proximal and distal balloons 168, 172 may be made of a pliable material such as nylon, PET, urethane, or other suitable material that allows inflation with injection of a fluid or air but is strong enough to withstand the pressure applied by blood flow. The diameter of the balloons 168, 172, when fully inflated, generally may vary between about 5 millimeters (mm) and about 30 mm, thereby allowing for occlusion and treatment of various sizes of blood vessels. Further, each of the balloons 168, 172 may include a marker configured to allow for radiographic visualization. The markers may be made of steel, platinum, gold, or other appropriate radio-opaque material, without limitation.

Moreover, in some embodiments, the proximal and distal balloons 168, 172 may have different sizes to provide blood occlusion for junctional injuries where variably sized vessels require occlusion (e.g., an iliac injury at the level of the aortic bifurcation). Further, the inflation tubes 204, 208 preferably are made of a similar material to the balloons 168, 172, such as polyethylene, Teflon, or similar suitable material, without limitation.

While the double balloon occlusion catheter and methods have been described in terms of particular variations and illustrative figures, those of ordinary skill in the art will recognize that the double balloon occlusion catheter is not limited to the variations or figures described. In addition, where methods and steps described above indicate certain events occurring in certain order, those of ordinary skill in the art will recognize that the ordering of certain steps may be modified and that such modifications are in accordance with the variations of the double balloon occlusion catheter. Additionally, certain of the steps may be performed concurrently in a parallel process when possible, as well as performed sequentially as described above. To the extent there are variations of the double balloon occlusion catheter, which are within the spirit of the disclosure or equivalent to the double balloon occlusion catheter found in the claims, it is the intent that this patent will cover those variations as well. Therefore, the present disclosure is to be understood as not limited by the specific embodiments described herein, but only by scope of the appended claims.

What is claimed is:

1. A vascular occlusion catheter, comprising:
a catheter for reaching an injury site within an artery;
a proximal balloon for being inflated at a first side of the injury site;
a distal balloon coupled for being inflated at a second side of the injury site; and
a blood occlusion segment disposed between the proximal balloon and the distal balloon.

2. The vascular occlusion catheter of claim 1, wherein the catheter includes a shunt that extends through the proximal balloon and the distal balloon to maintain blood flow through the artery.

3. The vascular occlusion catheter of claim 1, wherein the proximal balloon and the distal balloon are disposed near a distal aspect of the catheter with a space between the proximal balloon and the distal balloon to provide the blood occlusion segment of the catheter when the proximal balloon and the distal balloon are inflated.

4. The vascular occlusion catheter of claim 1, wherein one or more of the proximal balloon and the distal balloon include a marker for radiographic visualization.

5. The vascular occlusion catheter of claim 1, wherein the catheter includes a central lumen for receiving a wire to guide the catheter to the injury site.

6. The vascular occlusion catheter of claim 5, wherein a luer-lock is disposed at a proximal aspect of the catheter for providing access to the central lumen for flushing and wire entry.

7. The vascular occlusion catheter of claim 5, wherein a first luer-lock and a second luer-lock are disposed near a proximal aspect of the catheter for respectively inflating the proximal balloon and the distal balloon.

8. The vascular occlusion catheter of claim 7, wherein a first inflation tube is disposed in the central lumen and provides fluid communication between the first luer-lock and the proximal balloon.

9. The vascular occlusion catheter of claim 7, wherein a second inflation tube is disposed in the central lumen and provides fluid communication between the second luer-lock and the distal balloon.

10. The vascular occlusion catheter of claim 5, wherein a side port is disposed near a proximal aspect of the catheter and configured to facilitate flushing the central lumen and injecting contrast dye.

11. The vascular occlusion catheter of claim 1, wherein the catheter includes proximal shunt perforations that are proximal of the proximal balloon and configured to allow blood to enter a central lumen of the catheter and exit through an open distal end of the catheter.

12. The vascular occlusion catheter of claim 11, wherein the catheter includes distal shunt perforations that are distal of the distal balloon and configured to allow blood to enter or exit a bidirectional lumen of the catheter.

13. The vascular occlusion catheter of claim 12, wherein the bidirectional lumen is concentric with a central lumen and provides fluid communication between the distal shunt perforations and the proximal shunt perforations.

14. The vascular occlusion catheter of claim 13, wherein the bidirectional lumen is configured to cause blood flow to bypass the blood occlusion segment.

15. A method for vascular occlusion, comprising:
navigating a wire to an injury site within an artery;
crossing the injury site with the wire;
advancing a catheter over the wire to the injury site;
positioning a proximal balloon at a first side of the injury site;
positioning a distal balloon at a second side of the injury site; and
inflating the proximal balloon and inflating the distal balloon to establish a blood occlusion segment at the injury site.

16. The method of claim 15, wherein positioning the proximal balloon and positioning the distal balloon include using radio-opaque marker to indicate the centers of the proximal balloon and the distal balloon within the artery.

17. The method of claim 15, wherein inflating the proximal balloon and inflating the distal balloon includes causing unobstructed blood flow to bypass the blood occlusion segment.

18. The method of claim 17, wherein causing unobstructed blood flow includes directing the blood flow through proximal shunt perforations into a central lumen of the catheter.

19. The method of claim 17, wherein causing unobstructed blood flow includes directing the blood flow through a bidirectional lumen comprising the catheter.

20. The method of claim 19, wherein directing the blood flow includes allowing blood to flow through proximal shunt perforations and distal shunt perforations that are in fluid communication with the bidirectional lumen.
